(19) United States
(12) Patent Application Publication
(10) Pub. No.: US 2003/0187283 A1
(43) Pub. Date: Oct. 2, 2003

(54) Catalyst

(76) Inventors: Ursula Jansen, Neuss (DE); Andreas Wegner, Mulheim (DE); Markus Dugal, Koln (DE)

Correspondence Address: BAYER POLYMERS LLC, 100 BAYER ROAD, PITTSBURGH, PA, 15205, US

(21) Appl. No.: 10/372,360

(22) Filed: Feb. 21, 2003

(30) Foreign Application Priority Data
Feb. 26, 2002 (DE) 10208254.5

(51) Int. Cl. C07D 301/03

(52) U.S. Cl. 549/523; 502/304; 502/324; 502/345; 502/325

(57) ABSTRACT

The present invention provides a catalyst for the epoxidation of hydrocarbons with oxygen, a process for the preparation of the catalyst, and a process for the epoxidation of hydrocarbons with oxygen in the presence of the catalyst.

### FIELD OF THE INVENTION

The present invention provides a catalyst for the epoxidation of hydrocarbons with oxygen, a process for the preparation of the catalyst, and a process for the epoxidation of hydrocarbons with oxygen in the presence of the catalyst.

### BACKGROUND OF THE INVENTION

Epoxides are an important starting material for the polyurethane industry. There are a number of processes for their preparation, some of which have also been converted to a commercial scale. The industrial manufacture of ethylene oxide is effected, for example, by the direct oxidation of ethene with air or with gases containing molecular oxygen, in the presence of a catalyst containing silver. That process is described in EP-A 0 933 130.

To prepare longer-chain epoxides, hydrogen peroxide or hypochlorite is generally used on a commercial scale as the oxidizing agent in the liquid phase. EP-A 0 930 308 describes the use of ion-exchanged titanium silicalites as catalyst with those two oxidizing agents.

A further class of oxidation catalysts, allowing the oxidation of propene in the gas phase to the corresponding epoxide (propene oxide abbreviated herein as PO), is disclosed in U.S. Pat. No. 5,623,090. In that process, gold is used on anatase as catalyst. The oxidizing agent used is oxygen, which is employed in the presence of hydrogen. The system is distinguished by extraordinarily high selectivity (S>95%) in respect of the propene oxidation. Disadvantages are the low conversion and the deactivation of the catalyst, as well as the high consumption of hydrogen.

Some mixtures of elements of groups 3 to 10 and 14 to 16 of the periodic system according to IUPAC (definition of 1986) are known in the art as catalysts for other processes. For example, mixtures of iron, cobalt and nickel on various supports are used in the preparation of ammonia. Reference is here made by way of example to the publication of M. Appl (M. Appl in Indian Chem. Eng., 1987, pages 7 to 29). Mixtures of iron and cobalt are also used in the oxidation of cyclohexane to adipic acid. That is disclosed in U.S. Pat. No. 5,547,905. The formation of epoxides is not disclosed.

DE-A 100 24 096 discloses that it is possible, using mixtures of various elements from the group Cu, Ru, Rh, Pd, Os, Ir, Pt, Au, In, Tl, Mn and Ce as catalyst, to prepare propene oxide by direct oxidation of propene with oxygen or air. It is unusual, in that process, that the oxidation stops at the epoxide stage and the corresponding acids, ketones or aldehydes are not formed.

DE-A 101 39 531 discloses that propene can be oxidized to propene oxide using as catalyst mixtures of various elements from the group Sc, Y, Ti, Zr, Hf, V, Nb, Ta, Cr, Mo, W, Re, Fe, Co, Ni, Sn, Pb, Sb, Bi and Se on a support.

The catalysts known from the art do not exhibit satisfactory results in respect of the activity of the direct oxidation of propene to propene oxide.

Direct oxidation is the oxidation of propene with oxygen or with gases containing oxygen.

It is important that the oxidation does not continue to the corresponding acid or to the aldehyde or ketone, but terminates at the epoxide stage.

### SUMMARY OF THE INVENTION

The present invention, therefore, provides catalysts that permit the direct oxidation of propene to propene oxide with a high level of activity.

### DETAILED DESCRIPTION OF THE INVENTION

The present invention will now be described for purposes of illustration and not limitation.

The present invention provides a catalyst containing a mixture of at least one element selected from the group consisting of Sc, Y, Ti, Zr, Hf, V, Nb, Ta, Cr, Mo, W, Re, Fe, Co, Ni, Sn, Pb, Sb, Bi, Se and Zn and at least one element selected from the group consisting of Cu, Ru, Rh, Pd, Os, Ir, Pt, Au, In, Tl, Mn and Ce, the mixture being on a porous support.

The porous support has a large specific surface area. The specific surface area can be measured, for example, according to the BET method. The BET surface area of the support is preferably less than 200 m²/g, particularly preferably less than 100 m²/g, before application of the mixture thereto.

The BET surface area of the support is preferably <200 m²/g, more preferably <100 m²/g, particularly preferably <10 m²/g. The BET surface area of the support is most preferably >1 m²/g.

The BET surface area is determined in the conventional manner. That determination is disclosed, for example, in the publication of Brunauer, Emmet and Teller in J. Anorg. Chem. Soc. 1938, Volume 60, page 309.

The elements may be present in the mixture in elemental form or in the form of chemical compounds.

The elements are preferably present in the form of oxides or in the form of hydroxides or in elemental form.

The content of the elements on the support is preferably from 0.001 to 50 wt. %, particularly preferably from 0.001 to 20 wt. % and most preferably from 0.01 to 10 wt. %. The concentration data are based on the support.

The relative proportions of the elements can be varied within a wide range.

Also preferred is the catalyst in which the support contains Al₂O₃, CaCO₃, ZrO₂, SiO₂, SiC, TiO₂ or SiO₂—TiO₂ mixed oxide.

Also preferred is the catalyst in which the support consists of Al₂O₃, CaCO₃, SiO₂, ZrO₂, SiC, TiO₂ or SiO₂—TiO₂.

Also preferred is the catalyst in which the choice of elements from the two mentioned groups is made in such a manner that the mentioned mixture is selected from the group consisting of Bi—Rh, Bi—Ru, Cr—Cu, Cr—Ru, Fe—Ru, Fe—Tl, Fe—Cu, Sb—Ru, Sb—Cu, Ni—Ru, Mo—Cu, Ni—Rh, Ru—Re, Co—Ru, Co—Tl, Mn—Pb, Mn—Cu—Ag—Pb—In, Mn—Cu—Ag—Pb—Sr, Mn—Cu—Ag—Pb, Mn—Pb—Cu—Ru, Mn—Ru—Co—Ba, Eu—Ag—Ni—Tl, Mn—Cu—Ag—Zn, Mn—Ni—Ag—Pb, Mn—Pb—La—Cu, In—Mn—Pb—Ag, Mn—Co—Ag—Pb, Cs—Mn—Pb—Tl, Mn—Pb—Tl—Cu—Ag, Mn—Pb—Tl—Cu, Cs—Mn—Pb—Tl—Ag, Mn—Cu—Pb, Mn—Pb—Ag—Ru, Co—Mn—Pb—Cu—Ag, Co—Fe—Mn—Pb—Ag, Ce—Co—Mn—Pb—Ag, Co—In—Mn—Pb—Ag, Ce—In—Mn—Pb—Cu, and any desired combination of the mentioned mixtures.

The mentioned catalysts are provided by the present invention.

The catalysts according to the invention have high selectivity in respect of organic products in the oxidation of propene to propene oxide. They are also suitable for the epoxidation of other hydrocarbons.

The present invention also provides a process for the preparation of the catalyst according to the invention, comprising

a) preparing the support,

b) combining the support with a solution containing at least one element selected from the group consisting of Sc, Y, Ti, Zr, Hf, V, Nb, Ta, Cr, Mo, W, Re, Fe, Co, Ni, Sn, Pb, Sb, Bi, Se and Zn and at least one element selected from the group consisting of Cu, Ru, Rh, Pd, Os, Ir, Pt, Au, In, Tl, Mn and Ce, whereby a support loaded with the elements is obtained, and

c) calcining the support loaded with the elements at a temperature of from 200 to 1,000° C., preferably 400 to 1000° C., preferably in air or in the presence of reducing gases.

The elements are present in the solution in the form of compounds of the elements. Preference is given to organic or inorganic salts, preferably carboxylates, alcoholates, formiates, nitrates, carbonates, halides, phosphates, sulfates or acetylacetonates. Nitrates or carboxylates are particularly preferred.

It is also possible for two or more solutions to be supplied separately.

After the support has been combined with the solution, any excess solution can be separated off or concentrated by drying. The so-called incipient wetness process is preferably used.

The incipient wetness process is understood to mean the addition of a solution containing soluble element compounds to the support, the volume of the solution on the support being less than or equal to the pore volume of the support. The support accordingly remains macroscopically dry. As solvents for incipient wetness there may be used any solvents in which the element precursors are soluble, such as water, alcohols, (crown) ethers, esters, ketones, halogenated hydrocarbons, etc.

Where the compounds of the elements are sufficiently soluble, it may also be advantageous to use more solution volume and to concentrate the excess solution by drying. The good solubility of the compounds of the elements in that case ensures that no precipitation of solids occurs before the solution volume has been concentrated to the pore volume of the support. An effect comparable to that of the incipient wetness process is thereby achieved.

Preference is given to the process in which the support is combined with the solution in such a manner that the volume of the solution is less than or at most equal to the pore volume of the support.

One embodiment of the present invention is a process in Which drying is carried out before the calcination.

Another embodiment of the present invention is a process in which reduction is carried out after the calcination.

A further embodiment of the present invention is a catalyst obtainable according to the described process.

The present invention also provides a method of using the catalyst according to the invention as a catalyst for the epoxidation of hydrocarbons.

An embodiment of the present invention is a process for the epoxidation of hydrocarbons with oxygen in the presence of the catalyst according to the invention.

Another embodiment of the present invention is a process in which the hydrocarbon is selected from the group consisting of propene and butene.

The term hydrocarbon is understood to mean unsaturated or saturated hydrocarbons, such as olefins or alkanes, which may also contain hetero atoms such as N, O, P, S or halogens. The hydrocarbon may be acyclic, monocyclic, bicyclic or polycyclic. It may be monoolefinic, diolefinic or polyolefinic. In the case of hydrocarbons having two or more double bonds, the double bonds may be present in conjugated and non-conjugated form.

Preference is given to hydrocarbons from which there are formed oxidation products whose partial pressure at the reaction temperature is sufficiently low to remove the product from the catalyst continuously.

Preference is given to unsaturated and saturated hydrocarbons having from 2 to 20 carbon atoms, preferably from 3 to 10 carbon atoms, especially propene, propane, isobutane, isobutylene, 1-butene, 2-butene, cis-2-butene, trans-2-butene, 1,3-butadiene, pentene, pentane, 1-hexene, 1-hexane, hexadiene, cyclohexene and benzene.

The oxygen may be used in many different forms, such as molecular oxygen, air and nitrogen oxide. Molecular oxygen is preferred.

Suitable mixtures are especially binary, ternary, quaternary and quintary mixtures containing at least one element selected from the group consisting of Sc, Y, Ti, Zr, Hf, V, Nb, Ta, Cr, Mo, W, Re, Fe, Co, Ni, Sn, Pb, Sb, Bi, Se and Zn and, at the same time, at least one element selected from the group Cu, Ru, Rh, Pd, Os, Ir, Pt, Au, In, Tl, Mn, Ce.

The supports are preferably compounds selected from the group consisting of Al₂O₃, SiO₂, CeO₂, ZrO₂, SiC, TiO₂, alkylsilicon oxides of the formula R—SiO_{1.5} wherein R=alkyl (especially methyl), and mixtures of the mentioned compounds.

The porosity of the support is advantageously from 20 to 60%, especially from 30 to 50%.

The particle size of the supports is dependent on the process conditions of the gas-phase oxidation and is usually in the range from 1/10 to 1/20 of the diameter of the reactor.

The porosity of the support is determined by mercury porosimetry, and the particle size of the element particles on the surface of the support is determined by means of electron microscopy and X-ray diffractometry.

The process for preparing the mixture of the elements on the support is not limited to one process. Mention may be made here of some examples of processes for generating element particles, such as deposition-precipitation, as described on page 3, line 38 ff of EP-B 0 709 360, or impregnation in solution, or incipient wetness processes, or colloid processes, or sputtering, or CVD, or PVD (CVD: chemical vapor deposition; PVD: physical vapor deposition).

The support is preferably impregnated with a solution containing the element ions and then dried and reduced. The solution may additionally contain components known to those skilled in the art, which components are able to increase the solubility of the element salt(s) in the solvent and/or change the redox potential of the elements and/or change the pH value. Special mention may be made of ammonia, amines, diamines, hydroxyamines and acids, such as HCl, HNO₃, H₂SO₄, H₃PO₄.

Impregnation of the support with the solution can be carried out, for example, by the incipient wetness process, but is not limited thereto. The incipient wetness process may comprise the following steps:

coating once with one element and/or coating several times with a different element,

coating once with some of the elements or with all the elements in one step,

coating several times with several elements in one or more steps in succession,

coating several times with several elements alternately in one or more steps.

Drying takes place preferably at a temperature of from approximately 40 to approximately 200° C. at normal pressure or, alternatively, reduced pressure. At normal pressure, it is possible to work under an atmosphere of air or, alternatively, under an inert gas atmosphere (e.g. Ar, N₂, He). The drying time is preferably in the range from 2 to 24 hours, preferably from 4 to 8 hours.

The calcination preferably takes place either under an inert gas atmosphere and subsequently or solely under an oxygen-containing gas atmosphere. The oxygen contents in the gas stream are advantageously in the range from 0 to 21 vol. %, preferably from 5 to 15 vol. %. The temperature for the calcination is adapted to the element mixture and is therefore generally in the range from 200 to 1000° C., preferably from 400 to 800° C., more preferably from 450 to 550° C., most preferably 500° C.

The reduction takes place preferably at elevated temperatures under a hydrogen-containing nitrogen atmosphere. The content of hydrogen may be from 0 to 100 vol. %. It is preferably from 0 to 25 vol. %, particularly preferably 10 vol. %. The reduction temperatures are adapted to the element mixture in question and are preferably from 100 to 800° C.

It may be advantageous to add to the element mixture conventional promoters or moderators, such as alkaline earth and/or alkali ions in the form of hydroxides, carbonates, nitrates, chlorides of one or more alkaline earth and/or alkali elements and/or silver. These are described, for example, on page 4, line 39 ff of EP-A 0 933 130.

The epoxidation process is preferably carried out in the gas phase under the following conditions.

The molar amount of hydrocarbon used relative to the total number of moles of hydrocarbon, oxygen and, optionally, diluent gas, as well as the relative molar ratio of the components, can be varied within wide ranges and is generally governed by the explosive limits of the hydrocarbon-oxygen mixture. In general, the reaction is carried out above or below the explosive limits.

An excess of hydrocarbon, relative to the oxygen used (on a molar basis), is preferably employed. The hydrocarbon content in the oxygen is typically ≦2 mol % and ≧78 mol %. The chosen hydrocarbon contents are preferably in the range from 0.5 to 2 mol % in the case of procedures below the explosive limit, and from 78 to 99 mol % in the case of procedures above the explosive limit. The ranges from 1 to 2 mol % and from 78 to 90 mol %, respectively, are particularly preferred.

The molar amount of oxygen, relative to the total number of moles of hydrocarbon, oxygen and diluent gas, can be varied within wide ranges. The molar amount of oxygen used is preferably lower, relative to the hydrocarbon. Preference is given to the use of amounts of oxygen in the range from 1 to 21 mol %, particularly preferably from 5 to 21 mol %, relative to the hydrocarbon.

In addition to the hydrocarbon and oxygen, it is optionally possible also to use a diluent gas, such as nitrogen, helium, argon, methane, carbon dioxide, carbon monoxide, perfluoropropane or similar, predominantly inert gases. Mixtures of the described inert components may also be used. The addition of inert components is beneficial for transporting away the heat freed in the exothermic oxidation reaction, and from the point of view of safety. In that case, it is possible for the above-described composition of the starting gas mixtures to be in the explosive range. A preferred range for a procedure with nitrogen as diluent gas is from 5 to 30 mol % in respect of hydrocarbon, from 50 to 75 mol % in respect of nitrogen and from 5 to 21 mol % in respect of oxygen.

Instead of a mixture of pure gases, air may also be used as the oxidizing agent. The amount of hydrocarbon in air is typically in a range from 5 to 50 mol %, preferably in a range from 15 to 25 mol %.

The contact time of hydrocarbon and catalyst is generally in the range from 5 to 60 seconds.

The process is generally carried out at temperatures in the range from 120 to 300° C., preferably from 150 to 260° C., particularly preferably from 170 to 230° C.

### EXAMPLES

In the Examples, as elsewhere in the present text, PO stands for propylene oxide.

### Examples of the Preparation of Catalysts and Testing Thereof in a Continuously Operating Fixed-Bed Reactor

### General Procedure Examples 1 to 30

Solution 1 is first prepared (see Table I), added to approximately 10 g of Al₂O₃ and is left to be absorbed. The solid so obtained is dried for 4 hours at 100° C. in a vacuum drying cabinet under a vacuum of approximately 15 mm Hg. Solution 2 is then left to be absorbed completely by the solid. The solid is dried overnight at 100° C. in a vacuum drying cabinet under a vacuum of approximately 15 mm Hg.

Finally, the precursor so prepared is reduced for 8 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. The results are given in Table I.

TABLE I
Preparation of solutions 1 and 2, results

| | | | | | | PO | |
|---|---|---|---|---|---|---|---|
| | | | | | | content | |
| | Solution 1 | | Solution 2 | | | ppm in | |
| | | Solvent | | Solvent | Internal | the | |
| | Element salt | (weighed | Element salt | (weighed | temp. | waste | Selectivity |
| Ex. | (weighed portion) | portion) | (weighed portion) | portion) | ° C. | gas | number* |
| 1 | antimony pentachloride | EtOH | hexachloroiridium | H₂O | 160 | 33 | <1 |
| | 646 mg | 3.8 g | solution (23%) 2 g | 3.5 g | | | |
| 2 | Bi(OOCCH(C₂H₅)C₄H₉)₃ | EtOH | ruthenium nitrosylnitrate | H₂O | 220 | 390 | 3.64 |
| | 90 mg | 3.8 g | solution | 1.5 g | | | |
| | | | (13.9%) 3.6 g | | | | |
| 3 | chromium nitrate | H₂O | ruthenium nitrosylnitrate | H₂O | 200 | 340 | <1 |
| | 2.02 g | 4 g | solution | 3.5 g | | | |
| | | | (13.9%) 1.91 g | | | | |
| 4 | chromium nitrate | H₂O | silver nitrate | H₂O | 210 | 110 | <1 |
| | 2.02 g | 4 g | 414.2 mg | 4.5 g | | | |
| 5 | chromium nitrate | H₂O | copper nitrate | H₂O | 230 | 130 | <1 |
| | 2.02 g | 4 g | 776.2 mg | 4 g | | | |
| 6 | chromium nitrate | H₂O | rhodium nitrate | — | 185 | 116 | <1 |
| | 2.02 g | 4 g | solution (10%) 7.76 g | | | | |
| 7 | iron nitrate | H₂O | ruthenium nitrosylnitrate | H₂O | 220 | 260 | 3.3 |
| | 1.902 g | 3.5 g | solution | 3.5 g | | | |
| | | | (13.9%) 1.91 g | | | | |
| 8 | iron nitrate | H₂O | copper nitrate | H₂O | 240 | 188 | <1 |
| | 1.902 g | 3.5 g | 776.2 mg | 4 g | | | |
| 9 | iron nitrate | H₂O | thallium nitrate | H₂O | 250 | 177 | 5.0 |
| | 190 mg | 4.5 g | 1.302 g | 4.5 g | | | |
| 10 | iron nitrate | H₂O | manganese nitrate | H₂O | 230 | 40 | <1 |
| | 190 mg | 4.5 g | 2.283 g | 4.5 g | | | |
| 11 | antimony pentachloride | EtOH | ruthenium nitrosylnitrate | H₂O | 200 | 245 | <1 |
| | 646 mg | 3.8 g | solution | 3.5 g | | | |
| | | | (13.9%) 1.91 g | | | | |
| 12 | antimony pentachloride | EtOH | copper nitrate | H₂O | 230 | 272 | <1 |
| | 64.6 mg | 3.8 g | 1.474 g | 3.5 g | | | |
| 13 | nickel nitrate | H₂O | ruthenium nitrosylnitrate | H₂O | 210 | 245 | <1 |
| | 1.3 g | 4 g | solution | 3.5 g | | | |
| | | | (13.9%) 1.91 g | | | | |
| 14 | cobalt nitrate | H₂O | ruthenium nitrosylnitrate | H₂O | 210 | 385 | <1 |
| | 2.467 g | 3 g | solution | 4.5 g | | | |
| | | | (13.9%) 0.191 g | | | | |
| 15 | cobalt nitrate | H₂O | thallium nitrate | H₂O | 230 | 316 | 3.8 |
| | 1.298 g | 4 g | 0.68 g | 4.5 g | | | |
| 16 | cobalt nitrate | H₂O | copper nitrate | H₂O | 230 | 218 | <1 |
| | 1.298 g | 4 g | 0.776 g | 4 g | | | |
| 17 | cobalt nitrate | H₂O | hexachloroiridium | H₂O | 195 | 76 | <1 |
| | 2.468 g | 3 g | solution (23%) 0.2 g | 4.5 g | | | |
| 18 | cobalt nitrate | H₂O | cerium nitrate | H₂O | 220 | 55 | <1 |
| | 2.468 g | 3 g | 81.5 mg | 4.5 g | | | |
| 19 | cobalt nitrate | H₂O | indium nitrate | H₂O | 230 | 60 | <1 |
| | 2.467 g | 3 g | 69 mg | 4.5 g | | | |
| 20 | cobalt nitrate | H₂O | rhodium nitrate | — | 175 | 153 | <1 |
| | 0.129 g | 4.5 g | solution (10%) | | | | |
| | | | 14.029 g | | | | |
| 21 | cobalt nitrate | H₂O | palladium nitrate | H₂O | 215 | 46 | <1 |
| | 2.468 g | 3 g | 56.9 mg | 4.5 g | | | |
| 22 | molybdenum oxychloride | EtOH | copper nitrate | H₂O | 220 | 145 | <1 |
| | 0.546 g | 3.8 g | 776.2 mg | 4 g | | | |
| 23 | Bi(OOCCH(C₂H₅)C₄H₉)₃ | EtOH | rhodium nitrate | — | 200 | 160 | <1 |
| | 90 mg | 3.8 g | solution (10%) | | | | |
| | | | 14.029 g | | | | |
| 24 | Bi(OOCCH(C₂H₅)C₄H₉)₃ | EtOH | copper nitrate | H₂O | 230 | 150 | <1 |
| | 90 mg | 3.8 g | 1.474 g | 3.5 g | | | |
| 25 | Bi(OOCCH(C₂H₅)C₄H₉)₃ | EtOH | thallium nitrate | H₂O | 230 | 27 | <1 |
| | 90 mg | 3.8 g | 1.302 g | 4.5 g | | | |
| 26 | nickel nitrate | H₂O | rhodium nitrate | — | 220 | 169 | <1 |
| | 1.303 g | 4 g | solution (10%) 7.76 g | | | | |
| 27 | nickel nitrate | H₂O | copper nitrate | H₂O | 225 | 111 | <1 |
| | 1.303 g | 4 g | 776.2 mg | 4 g | | | |
| 28 | ruthenium nitrosylnitrate | H₂O | rhenium acid | H₂O | 230 | 192 | 3.35 |
| | solution (13.9%) 3.631 g | 1.5 g | (59%) 45 mg | 4.5 g | | | |
| 29 | rhenium acid | H₂O | rhodium nitrate | — | 195 | 139 | <1 |
| | (62%) 42 mg | 4.5 g | solution (10%) | | | | |
| | | | 14.029 g | | | | |
| 30 | thallium nitrate | H₂O | rhenium acid | H₂O | 230 | 127 | n.d. |
| | 0.68 g | 4.5 g | (59%) 446 mg | 4.5 g | | | |

*Selectivity number = PO/Σ(organic products)

The following comparative examples serve for comparison with Examples 31 to 47. The comparative examples do not fulfill the conditions that at least one element was selected from each of the groups according to the invention.

### Comparative Example 1

One possible method of preparing an active catalyst for PO production consists in dissolving 77.6 mg of copper nitrate and 3.59 g of an approximately 14% ruthenium nitrosylnitrate solution in 2 ml of water, adding the solution to approximately 10 g of Al₂O₃ and leaving it to be absorbed. The solid so obtained is dried overnight at 100° C. in a vacuum drying cabinet under a vacuum of approximately 15 mm Hg.

Finally, the precursor so prepared is reduced for 12 hours at 500° C. with 10 vol. % H₂ in N₂ at 60 l/h.

After the reduction, 10 g of the catalyst so obtained are tested in a continuously operating fixed-bed reactor with a dwell time of approximately 20 seconds and a starting gas composition of 79 vol. % propene and 21 vol. % oxygen. At an internal temperature of 217° C., PO contents of 680 ppm are determined in the waste-gas stream.

### Comparative Example 2

Another possible method of preparing an active catalyst for PO production consists in dissolving 77.6 mg of copper nitrate in 5-6 ml of water, adding the solution to approximately 10 g of Al₂O₃ and leaving it to be absorbed. The solid so obtained is dried for 12 hours at 60° C. in a vacuum drying cabinet under a vacuum of approximately 15 mm Hg. Coating is then carried out in the same manner 6 times with a ruthenium nitrosylnitrate solution containing approximately 1.5 wt. % Ru, according to the absorptive capacity of the support. Drying is carried out as above for 4 hours between each of the coating operations.

Finally, the precursor so prepared is reduced for 12 hours at 500° C. with 10 vol. % H₂ in N₂ at 60 l/h.

After the reduction, 10 g of the catalyst so obtained are tested in a continuously operating fixed-bed reactor with a dwell time of approximately 20 seconds and a starting gas composition of 79 vol. % propene and 21 vol. % oxygen. At an internal temperature of 200° C., PO contents of 300 ppm are determined in the waste-gas stream.

### Comparative Example 3

An additional possible method of preparing an active catalyst for PO production consists in adding 7.4 g of a 10% rhodium nitrate solution to approximately 10 g of Al₂O₃ and leaving the solution to be absorbed. The solid so obtained is dried for 4 hours at 100° C. in a vacuum drying cabinet under a vacuum of approximately 15 mm Hg. Coating is then carried out in the same manner with 1.3 g of a ruthenium nitrosylnitrate solution containing approximately 20 wt. % Ru, and drying is then carried out for 12 hours as described in a vacuum drying cabinet. Finally, the precursor so prepared is reduced for 4 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of approximately 199° C., PO contents of 360 ppm are determined in the waste-gas stream.

### Comparative Example 4

An alternative possible method of preparing an active catalyst for PO production consists in dissolving 343 mg of thallium nitrate in 5 g of water and impregnating approximately 10 g of Al₂O₃ with the solution so formed. The solution is left to be absorbed, with constant agitation, and the solid so obtained is dried for 4 hours at 100° C. in a vacuum drying cabinet under a vacuum of approximately 15 mm Hg. Coating is then carried out in the same manner with a solution prepared from 776 mg of copper(II) nitrate and 5 g of water, and drying is then carried out overnight at 100° C. in a vacuum drying cabinet under approximately 15 mm Hg.

Finally, the precursor so prepared is reduced for 12 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 228° C., PO contents of 380 ppm are measured in the waste-gas stream.

### Comparative Example 5

2.5 g of a 20% ruthenium nitrosylnitrate solution are dissolved in 3 g of water, and 10 g of Al₂O₃ are impregnated with the solution so formed. The solution is left to be absorbed, with constant agitation, and the solid so obtained is dried for 4 hours at 100° C. in a vacuum drying cabinet under a vacuum of approximately 15 mm Hg. Coating is then carried out in the same manner with a solution prepared from 109 mg of 24% hexachloroiridium acid solution and 4.5 g of water, and drying is then carried out overnight at 100° C. in a vacuum drying cabinet under approximately 15 mm Hg.

Finally, the precursor so prepared is reduced for 12 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 208° C., PO contents of 540 ppm are measured in the waste-gas stream.

### Comparative Example 6

343 mg of thallium nitrate are dissolved in 5 g of water, and 10 g of Al₂O₃ are impregnated with the solution so formed. The solution is left to be absorbed, with constant agitation, and the solid so obtained is dried for 4 hours at 100° C. in a vacuum drying cabinet under a vacuum of approximately 15 mm Hg. Coating is then carried out in the same manner with a solution prepared from 1.3 g of a 20% ruthenium nitrosylnitrate solution and 4 g of water, and drying is then carried out overnight at 100° C. in a vacuum drying cabinet under approximately 15 mm Hg.

Finally, the precursor so prepared is reduced for 12 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 211° C., PO contents of 390 ppm are measured in the waste-gas stream.

### Comparative Example 7

17.86 g of copper nitrate are dissolved in 103 g of water, and 230 g of Al₂O₃ are impregnated with the solution so formed. The solution is left to be absorbed, with constant agitation, and the solid so obtained is dried for 4 hours at 100° C. in a vacuum drying cabinet under a vacuum of approximately 15 mm Hg. Coating is then carried out in the same manner with a solution prepared from 43.52 g of a 14% ruthenium nitrosylnitrate solution and 71 g of water, and drying is then carried out overnight at 100° C. in a vacuum drying cabinet under approximately 15 mm Hg.

The precursor so prepared is reduced for 4 hours at 500° C. with 10 vol. % H₂ in N₂ at 60 l/h.

5 g of the resulting solid are then coated with a solution prepared from 6 mg of palladium nitrate in 2.25 g of water, and drying is carried out overnight at 100° C. in a vacuum drying cabinet.

Finally, the precursor so prepared is reduced for 8 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 220° C., PO contents of 745 ppm are measured in the waste-gas stream.

### Comparative Example 8

2.76 g of manganese nitrate are dissolved in 103.5 g of water, and 230 g of Al₂O₃ are impregnated with the solution so formed. The solution is left to be absorbed, with constant agitation, and the solid so obtained is dried for 4 hours at 100° C. in a vacuum drying cabinet under a vacuum of approximately 15 mm Hg. Coating is then carried out in the same manner with a solution prepared from 33.92 g of copper nitrate and 95 g of water, and drying is then carried out overnight at 100° C. in a vacuum drying cabinet under approximately 15 mm Hg.

The precursor so prepared is reduced for 8 hours at 500° C. with 10 vol. % H₂ in N₂ at 60 l/h.

5 g of the resulting solid are then coated with a solution prepared from 6 mg of a 43.5% tetrachlorogold solution in 2.25 g of water, and drying is carried out overnight at 100° C. in a vacuum drying cabinet.

Finally, the precursor so prepared is reduced for 8 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 230° C., PO contents of 982 ppm are measured in the waste-gas stream.

### Examples of the Preparation of Catalysts and Testing Thereof in a Continuously Operating Fixed-Bed Reactor

### Examples 31 to 44

In the following Examples, element salt stock solutions were first prepared (Table II).

TABLE II
Preparation of aqueous element salt stock solutions

| Solution | Element salt | Amount [g] | Water [g] |
|---|---|---|---|
| S-1 | Manganese nitrate | 40.09 | 64.2 |
| S-2 | Copper nitrate | 25.9 | 73.5 |
| S-3 | Silver nitrate | 13.82 | 75.0 |
| S-4 | Lead acetate | 2.68 | 112.5 |
| S-5 | Cobalt nitrate | 23.11 | 35.2 |
| S-6 | Zinc nitrate | 39.93 | 60.0 |
| S-7 | Europium nitrate | 9.89 | 28.0 |
| S-8 | Nickel nitrate | 43.46 | 60.0 |
| S-9 | Thallium nitrate | 4.575 | 30.0 |
| S-10 | Lead acetate | 3.0 | 56.25 |
| S-11 | Lead acetate | 1.2 | 56.25 |
| S-12 | Trinitratonitrosylruthenium | 63.12 | 34.0 |
| | solution, 13.9% | | |
| S-13 | Barium chloride | 15.0 | 73.0 |
| S-14 | Indium nitrate | 2.76 | 90.0 |
| S-15 | Strontium nitrate | 0.19 | 67.5 |

The element salt stock solutions were then mixed in defined ratios by means of an automatic pipetting device (Table III and Table IV). The resulting solutions were then absorbed completely by 5 g of Al₂O₃. The solids so prepared were then dried overnight at 100° C. in a vacuum drying cabinet under a vacuum of approximately 15 mm Hg.

TABLE III

| | | | | | | PO |
|---|---|---|---|---|---|---|
| | | | | | | content |
| | | | | | | in the |
| | | | | | Reactor | waste |
| | Element salt stock solution/amount | | | | temp. | gas |
| Ex. | [no. from Table 1]/[μl] | | | | [° C.] | [ppm] |
| 31 | S-7/102 | S-3/1943 | S-8/102 | S-9/102 | 240 | 1990 |
| 32 | S-1/460 | S-2/92 | S-3/1748 | S-6/92 | 220 | 1171 |
| 33 | S-1/750 | S-8/750 | S-3/750 | S-4/313* | 230 | 1556 |
| 34 | S-1/205 | S-8/1023 | S-3/1023 | S-4/313* | 225 | 1385 |
| 35 | S-1/1023 | S-5/205 | S-3/1023 | S-4/313* | 215 | 2509 |
| 36 | S-1/90 | S-5/1710 | S-3/450 | S-4/313* | 220 | 2281 |
| 37 | S-1/90 | S-5/450 | S-3/1710 | S-4/313* | 230 | 3678 |
| 38 | S-1/1023 | S-2/205 | S-3/1023 | S-4/313* | 210 | 3173 |
| 39 | S-1/1607 | S-2/321 | S-3/321 | S-4/313* | 200 | 3057 |
| 40 | S-1/1474 | S-2/388 | S-3/388 | S-4/313* | 210 | 2814 |
| 41 | S-1/1688 | S-2/563 | S-10/2250* | | 210 | 1434 |
| 42 | S-1/563 | S-2/1688 | S-11/2250* | | 235 | 1819 |
| 43 | S-1/113 | S-2/2138 | S-11/2250* | | 250 | 1511 |
| 44 | S-1/747 | S-12/747 | S-5/747 | S-13/149 | 210 | 30 |

*Applied after coating and drying of the solid for 24 hours at 100° C. in a vacuum drying cabinet in a further step.

TABLE IV
Coating in three steps

| | | | | | | | PO |
|---|---|---|---|---|---|---|---|
| | | | | | | | content |
| | | | | | | React | in the |
| | | | | | | or | waste |
| | Element salt stock solution/amount | | | | | temp. | gas |
| Ex. | [no. from Table 1]/[μl] | | | | | [° C.] | [ppm] |
| 45 | S-1/1023 | S-2/205 | S-3/1023 | S-4/ | S-14/ | 210 | 2899 |
| | | | | 2500* | 2500* | | |
| 46 | S-1/1023 | S-2/205 | S-3/1023 | S-4/ | S-15/ | 210 | 2763 |
| | | | | 2500* | 2500* | | |

*Applied after coating and drying of the solid for 24 hours at 100° C. in a vacuum drying cabinet in a further step.

After the reduction, 1 g of the catalyst so obtained is tested in a continuously operating fixed-bed reactor with a dwell time of approximately 20 seconds and a starting gas composition of 79 vol. % propene and 21 vol. % oxygen. The results are shown in Tables III and Table IV.

### Example of the Preparation of Incipient Wetness Catalysts

### Example 47

A possible method of preparing an active catalyst for PO production consists in dissolving 5.39 g of manganese nitrate, 0.38 g of copper nitrate and 1.54 g of thallium nitrate in 23 g of water, adding the solution to approximately 50 g of Al₂O₃ and leaving it to be absorbed. The solid so obtained is dried for 24 hours at 100° C. in a vacuum drying cabinet under a vacuum of approximately 15 mm Hg.

0.24 g of lead acetate is then dissolved in 25 g of water, and the solution is left to be absorbed completely by the solid obtained previously. The resulting solid is then dried for 24 hours at 100° C. in a vacuum drying cabinet under a vacuum of approximately 15 mm Hg.

Finally, the precursor so prepared is reduced for 8 hours at 500° C. with 10 vol. % H₂ in N₂ at 60 l/h.

After the reduction, 1 g of the catalyst so obtained is tested in a continuously operating fixed-bed reactor with a dwell time of approximately 20 seconds and a starting gas composition of 79 vol. % propene and 21 vol. % oxygen. At an internal temperature of 230° C., PO contents of 1505 ppm are determined in the waste-gas stream.

### Example 48

Preparation of Incipient Wetness Catalysts on Various Support Materials and Test in a Continuously Operating Fixed-Bed Reactor

a) General Procedure for Al₂O₃-Supported Catalysts

In a 2 ml glass vessel, aqueous stock solutions of the various element precursors (see Table V, c=52.6 g/l based on the pure element) and promoters (see Table V, c=5.26 g/l) are combined by means of a number of from 1 to 5 micrometering pumps, according to the number of elements to be combined, from a corresponding number of storage vessels, so that the total metered volume of the solutions corresponds to approximately 450 μl. Where two or more elements and or promoters are combined, equal partial volumes of the different solutions are metered in (in the tables of results 5 to 11, the proportions of each of the element precursor solutions in the total metered volume are listed when indicating the composition).

Approximately 1 g of Al₂O₃ is added to the solution. Once the solution has been completely absorbed by the solid, the latter is dried overnight at approximately 100° C. and 200 mbar in a vacuum drying cabinet. The precursor so prepared is calcined for 4 hours at 500° C. in air and then transferred to a continuously operating fixed-bed reactor. After a conditioning phase of 4 hours at 200° C. in 10 vol. % H₂ in N₂ at 0.08 l/h, the catalyst is tested in a starting gas stream having the composition 24% propene/4.5% oxygen/71.5% air at a temperature of 200° C., at normal pressure and a flow rate of 0.35 l/h. The sample gas is tested at regular intervals by means of GC for propylene oxide formed (the results are given in Table VI).

b) General Procedure for ZrO₂-Supported Catalysts

In contrast to a), 1.3 g of ZrO₂ are added to the solution instead of Al₂O₃ (the results are given in Table VII).

c) General Procedure for CaCO₃-Supported Catalysts

In contrast to a), 1.0 g of CaCO₃ is added to the solution instead of Al₂O₃ (the results are given in Table VIII).

d) General Procedure for SiC-Supported Catalysts

In contrast to a), 0.6 g of SiC is added to the solution instead of Al₂O₃ (the results are given in Table IX).

e) General Procedure for SiO₂-Supported Catalysts

In contrast to a), 0.55 g of SiO₂ is added to the solution instead of Al₂O₃ (the results are given in Table X).

f) General Procedure for TiO₂-Supported Catalysts

In contrast to a), 1.0 g of TiO₂ is added to the solution instead of Al₂O₃ (the results are given in Table XI).

g) General Procedure for SiO₂—TiO₂-Supported Catalysts

In contrast to a), 0.5 g of TiO₂—SiO₂ mixed oxide is added to the solution instead of Al₂O₃ (the results are given in Table XII).

TABLE V
List of precursors used

| Substance name | Role | Formula symbol |
|---|---|---|
| Ammonium cerium(IV) nitrate | precursor | Ce |
| Cobalt(II) nitrate | precursor | Co |
| Chromium(III) nitrate | precursor | Cr |
| Iron(III) nitrate | precursor | Fe |
| Indium(III) nitrate | precursor | In |
| Manganese(II) nitrate | precursor | Mn |
| Ammonium heptamolybdate * 4 | precursor | Mo |
| H₂O | | |
| Lead(II) nitrate | precursor | Pb |
| Strontium nitrate | precursor | Sr |
| Samarium(II) acetate | precursor | Sm |
| Lanthanum nitrate | precursor | La |
| Copper(II) nitrate | precursor | Cu |
| Rhenium(VII) oxide | precursor | Re |
| Silver nitrate | precursor | Ag |
| Ruthenium nitrosylnitrate | precursor | Ru |
| Cobalt(II) nitrate | precursor | Co |
| Caesium nitrate | promoter | Cs |
| Iron(III) nitrate | precursor | Fe |
| Neodymium(III) nitrate | promoter | Nd |
| Potassium nitrate | promoter | K |
| Bismuth nitrate | precursor | Bi |
| Rhodium(III) nitrate | precursor | Rh |
| Palladium(II) nitrate | precursor | Pd |
| Tetramminplatinum(II) nitrate | precursor | Pt |
| Silver nitrate | precursor | Ag |
| Nickel(II) nitrate | precursor | Ni |
| Barium nitrate | promoter | Ba |
| Europium nitrate | promoter | Eu |
| Erbium(III) nitrate | promoter | Er |
| Yttrium(III) nitrate | promoter | Y |
| Sodium metatungstate | precursor | W |
| Thallium(III) nitrate | precursor | Tl |
| Niobium ammonium oxalate | precursor | Nb |
| Vanadium(III) chloride | precursor | V |
| Tin(II) chloride | precursor | Sn |

Table VI: Examples of Al₂O₃-Supported Catalysts

In the middle column of Table VI (composition), the proportion of the element precursor solutions in the total metered volume is given. The element symbol is always given followed by a number (e.g. Mn for manganese followed by 0.3333).

TABLE VI

| | | Propene to |
|---|---|---|
| | | PO |
| Ex- | | conversion |
| ample | Composition | [%] |
| 48-1 | Mn0.3333La0.3333Cu0.3333 | 0.005029 |
| 48-2 | Mn0.3333Pb0.3333Cu0.3333 | 0.024607 |
| 48-3 | Mn0.3333Pb0.3333La0.3333 | 0 |
| 48-4 | Mn0.3333Pb0.3333Sn0.3333 | 0 |
| 48-5 | Mn0.3333Pb0.3333V0.3333 | 0 |
| 48-6 | Mn0.3333Mo0.3333Pb0.3333 | 0 |
| 48-7 | In0.3333Mn0.3333Pb0.3333 | 0 |
| 48-8 | Fe0.3333Mn0.3333Pb0.3333 | 0.005131 |
| 48-9 | Cr0.3333Mn0.3333Cu0.3333 | 0.001775 |
| 48-10 | Cr0.3333Mn0.3333Pb0.3333 | 0 |
| 48-11 | Co0.3333Mn0.3333Pb0.3333 | 0.002914 |
| 48-12 | Ce0.3333Mn0.3333Pb0.3333 | 0 |
| 48-13 | Mn0.3333Pb0.3333Re0.3333 | 0 |
| 48-14 | Mn0.5Pb0.5 | 0.013806 |
| 48-15 | Mn0.5Pb0.5 | 0.002884 |
| 48-16 | Co0.5Ru0.5 | 0.006536 |
| 48-17 | Sm0.3333Ag0.3333Ru0.3333 | 0.001759 |
| 48-18 | Sm0.3333Re0.3333Ru0.3333 | 0.001754 |
| 48-19 | Sm0.3333Cu0.3333Re0.3333 | 0.001167 |
| 48-20 | Mn0.3333Sm0.3333Ru0.3333 | 0.001282 |
| 48-21 | Mn0.3333Sm0.3333Ag0.3333 | 0.00269 |
| 48-22 | Mn0.3333Sm0.3333Cu0.3333 | 0.007629 |
| 48-23 | Mn0.3333Pb0.3333Ag0.3333 | 0.006337 |
| 48-24 | Mn0.3333Pb0.3333Cu0.3333 | 0.015669 |
| 48-25 | Mn0.3333Pb0.3333Sm0.3333 | 0.001827 |
| 48-26 | Mn0.3333Pb0.3333Sr0.3333 | 0.001393 |
| 48-27 | Mn0.3333Mo0.3333Sm0.3333 | 0.001505 |
| 48-28 | In0.3333Sm0.3333Cu0.3333 | 0.001286 |
| 48-29 | Fe0.3333La0.3333Cu0.3333 | 0.002146 |
| 48-30 | Fe0.3333Sm0.3333Ru0.3333 | 0.001388 |
| 48-31 | Fe0.3333In0.3333Cu0.3333 | 0.003105 |
| 48-32 | Cr0.3333Sm0.3333Cu0.3333 | 0.001304 |
| 48-33 | Co0.3333Ag0.3333Ru0.3333 | 0.006319 |
| 48-34 | Co0.3333Cu0.3333Ag0.3333 | 0.006362 |
| 48-35 | Co0.3333Cu0.3333Re0.3333 | 0.001392 |
| 48-36 | Co0.3333La0.3333Ru0.3333 | 0.001302 |
| 48-37 | Co0.3333La0.3333Cu0.3333 | 0.001305 |
| 48-38 | Co0.3333Sm0.3333Cu0.3333 | 0.001158 |
| 48-39 | Co0.3333Mn0.3333Ru0.3333 | 0.009945 |
| 48-40 | Co0.3333In0.3333Ru0.3333 | 0.008738 |
| 48-41 | Co0.3333Cr0.3333Ru0.3333 | 0.001787 |
| 48-42 | Ce0.3333Sm0.3333Ru0.3333 | 0.001916 |
| 48-43 | Ce0.3333Sm0.3333Cu0.3333 | 0.002054 |
| 48-44 | Ce0.3333Co0.3333Ru0.3333 | 0.005344 |
| 48-45 | Ce0.3333Co0.3333Mn0.3333 | 0.00218 |
| 48-46 | Pb0.25Sm0.25Ag0.25Ru0.25 | 0.00492 |
| 48-47 | Pb0.25Sm0.25La0.25Ru0.25 | 0.004611 |
| 48-48 | Mo0.25Pb0.25Sm0.25Cu0.25 | 0.001481 |
| 48-49 | Mo0.25Pb0.25Sm0.25La0.25 | 0.002958 |
| 48-50 | Mn0.25Sm0.25Ag0.25Ru0.25 | 0.0135 |
| 48-51 | Mn0.25Sm0.25Re0.25Ag0.25 | 0.00163 |
| 48-52 | Mn0.25Sm0.25Cu0.25Ru0.25 | 0.006665 |
| 48-53 | Mn0.25Sm0.25Cu0.25Ag0.25 | 0.017459 |
| 48-54 | Mn0.25Sm0.25La0.25Ru0.25 | 0.006016 |
| 48-55 | Mn0.25Sm0.25La0.25Ag0.25 | 0.004829 |
| 48-56 | Mn0.25Sm0.25La0.25Cu0.25 | 0.006863 |
| 48-57 | Mn0.25Sr0.25Sm0.25Ru0.25 | 0.00525 |
| 48-58 | Mn0.25Sr0.25Sm0.25Ag0.25 | 0.003385 |
| 48-59 | Mn0.25Sr0.25Sm0.25Re0.25 | 0.002622 |
| 48-60 | Mn0.25Sr0.25Sm0.25Cu0.25 | 0.001748 |
| 48-61 | Mn0.25Sr0.25Sm0.25La0.25 | 0.00138 |
| 48-62 | Mn0.25Pb0.25Ag0.25Ru0.25 | 0.011533 |
| 48-63 | Mn0.25Pb0.25Re0.25Ru0.25 | 0.01587 |
| 48-64 | Mn0.25Pb0.25Re0.25Ag0.25 | 0.002148 |
| 48-65 | Mn0.25Pb0.25Cu0.25Ru0.25 | 0.018529 |
| 48-66 | Mn0.25Pb0.25Cu0.25Ag0.25 | 0.027683 |
| 48-67 | Mn0.25Pb0.25La0.25Ru0.25 | 0.009965 |
| 48-68 | Mn0.25Pb0.25La0.25Ag0.25 | 0.025801 |
| 48-69 | Mn0.25Pb0.25La0.25Cu0.25 | 0.01824 |
| 48-70 | Mn0.25Pb0.25Sm0.25Ru0.25 | 0.007951 |
| 48-71 | Mn0.25Pb0.25Sm0.25Ag0.25 | 0.016903 |
| 48-72 | Mn0.25Pb0.25Sm0.25La0.25 | 0.003864 |
| 48-73 | Mn0.25Pb0.25Sr0.25Ru0.25 | 0.009293 |
| 48-74 | Mn0.25Pb0.25Sr0.25Ag0.25 | 0.013894 |
| 48-75 | Mn0.25Pb0.25Sr0.25Re0.25 | 0.005076 |
| 48-76 | Mn0.25Pb0.25Sr0.25Cu0.25 | 0.016323 |
| 48-77 | Mn0.25Pb0.25Sr0.25La0.25 | 0.005412 |
| 48-78 | Mn0.25Pb0.25Sr0.25Sm0.25 | 0.001403 |
| 48-79 | Mn0.25Mo0.25Sm0.25Cu0.25 | 0.008523 |
| 48-80 | Mn0.25Mo0.25Sm0.25La0.25 | 0.002048 |
| 48-81 | Mn0.25Mo0.25Sr0.25Sm0.25 | 0.001704 |
| 48-82 | In0.25Pb0.25Sm0.25Ru0.25 | 0.00284 |
| 48-83 | In0.25Pb0.25Sr0.25Ru0.25 | 0.002701 |
| 48-84 | In0.25Mn0.25Sm0.25Ru0.25 | 0.003292 |
| 48-85 | In0.25Mn0.25Sm0.25Cu0.25 | 0.003912 |
| 48-86 | In0.25Mn0.25Pb0.25Ru0.25 | 0.005738 |
| 48-87 | In0.25Mn0.25Pb0.25Ag0.25 | 0.020141 |
| 48-88 | In0.25Mn0.25Pb0.25Cu0.25 | 0.018067 |
| 48-89 | In0.25Mn0.25Pb0.25La0.25 | 0.00214 |
| 48-90 | In0.25Mn0.25Pb0.25Sm0.25 | 0.005653 |
| 48-91 | In0.25Mn0.25Mo0.25Sm0.25 | 0.004851 |
| 48-92 | Fe0.25Pb0.25Sm0.25Ru0.25 | 0.002021 |
| 48-93 | Fe0.25Pb0.25Sm0.25Ag0.25 | 0.00473 |
| 48-94 | Fe0.25Pb0.25Sm0.25La0.25 | 0.001247 |
| 48-95 | Fe0.25Mn0.25Sm0.25Ru0.25 | 0.003598 |
| 48-96 | Fe0.25Mn0.25Sm0.25Ag0.25 | 0.005005 |
| 48-97 | Fe0.25Mn0.25Sm0.25Cu0.25 | 0.005536 |
| 48-98 | Fe0.25Mn0.25Pb0.25Ru0.25 | 0.003179 |
| 48-99 | Fe0.25Mn0.25Pb0.25Ag0.25 | 0.019248 |
| 48-100 | Fe0.25Mn0.25Pb0.25Cu0.25 | 0.014885 |
| 48-101 | Fe0.25Mn0.25Pb0.25La0.25 | 0.001479 |
| 48-102 | Fe0.25Mn0.25Pb0.25Sm0.25 | 0.002426 |
| 48-103 | Fe0.25In0.25Pb0.25Sm0.25 | 0.002377 |
| 48-104 | Cr0.25Pb0.25Sm0.25Ru0.25 | 0.001679 |
| 48-105 | Cr0.25Mn0.25Pb0.25Ru0.25 | 0.004468 |
| 48-106 | Cr0.25Mn0.25Pb0.25Cu0.25 | 0.002337 |
| 48-107 | Cr0.25Mn0.25Pb0.25La0.25 | 0.002503 |
| 48-108 | Fe0.2Mn0.2Cu0.2Ag0.2Ru0.2 | 0.011021 |
| 48-109 | Fe0.2Mn0.2Cu0.2Re0.2Ru0.2 | 0.001498 |
| 48-110 | Fe0.2Mn0.2Cu0.2Re0.2Ag0.2 | 0.00216 |
| 48-111 | Fe0.2Mn0.2La0.2Ag0.2Ru0.2 | 0.006401 |
| 48-112 | Fe0.2Mn0.2La0.2Cu0.2Ru0.2 | 0.009917 |
| 48-113 | Fe0.2Mn0.2La0.2Cu0.2Ag0.2 | 0.015484 |
| 48-114 | Fe0.2Mn0.2Sm0.2Ag0.2Ru0.2 | 0.004775 |
| 48-115 | Fe0.2Mn0.2Sm0.2Cu0.2Ru0.2 | 0.009364 |
| 48-116 | Fe0.2Mn0.2Sm0.2Cu0.2Ag0.2 | 0.009009 |
| 48-117 | Fe0.2Mn0.2Sm0.2La0.2Cu0.2 | 0.009655 |
| 48-118 | Fe0.2Mn0.2Sr0.2Cu0.2Ru0.2 | 0.007247 |
| 48-119 | Fe0.2Mn0.2Sr0.2Cu0.2Ag0.2 | 0.013507 |
| 48-120 | Fe0.2Mn0.2Sr0.2Cu0.2Re0.2 | 0.004994 |
| 48-121 | Fe0.2Mn0.2Sr0.2La0.2Ru0.2 | 0.004267 |
| 48-122 | Fe0.2Mn0.2Sr0.2La0.2Ag0.2 | 0.002531 |
| 48-123 | Fe0.2Mn0.2Sr0.2La0.2Cu0.2 | 0.010059 |
| 48-124 | Fe0.2Mn0.2Sr0.2Sm0.2Ru0.2 | 0.005028 |
| 48-125 | Fe0.2Mn0.2Sr0.2Sm0.2Ag0.2 | 0.00239 |
| 48-126 | Fe0.2Mn0.2Pb0.2Cu0.2Ru0.2 | 0.011371 |
| 48-127 | Fe0.2Mn0.2Pb0.2Cu0.2Ag0.2 | 0.019613 |
| 48-128 | Fe0.2Mn0.2Pb0.2Cu0.2Re0.2 | 0.006953 |
| 48-129 | Fe0.2Mn0.2Pb0.2La0.2Ag0.2 | 0.014571 |
| 48-130 | Fe0.2Mn0.2Pb0.2La0.2Cu0.2 | 0.020644 |
| 48-131 | Fe0.2Mn0.2Pb0.2Sm0.2Ru0.2 | 0.003301 |
| 48-132 | Fe0.2Mn0.2Pb0.2Sm0.2Ag0.2 | 0.009461 |
| 48-133 | Fe0.2Mn0.2Pb0.2Sm0.2Cu0.2 | 0.009229 |
| 48-134 | Fe0.2Mn0.2Pb0.2Sm0.2La0.2 | 0.002486 |
| 48-135 | Fe0.2Mn0.2Pb0.2Sr0.2Ru0.2 | 0.003154 |
| 48-136 | Fe0.2Mn0.2Pb0.2Sr0.2Ag0.2 | 0.007019 |
| 48-137 | Fe0.2Mn0.2Pb0.2Sr0.2Cu0.2 | 0.018365 |
| 48-138 | Fe0.2Mn0.2Mo0.2Cu0.2Ag0.2 | 0.009828 |
| 48-139 | Fe0.2Mn0.2Mo0.2La0.2Cu0.2 | 0.006754 |
| 48-140 | Fe0.2Mn0.2Mo0.2Sm0.2Ag0.2 | 0.00303 |
| 48-141 | Fe0.2Mn0.2Mo0.2Sm0.2Cu0.2 | 0.010538 |
| 48-142 | Fe0.2Mn0.2Mo0.2Pb0.2Ag0.2 | 0.009388 |
| 48-143 | Fe0.2Mn0.2Mo0.2Pb0.2Cu0.2 | 0.013733 |
| 48-144 | Fe0.2Mn0.2Mo0.2Pb0.2Sm0.2 | 0.001791 |
| 48-145 | Fe0.2In0.2Mn0.2Ag0.2Ru0.2 | 0.006129 |
| 48-146 | Fe0.2In0.2Mn0.2Cu0.2Ru0.2 | 0.004708 |
| 48-147 | Fe0.2In0.2Mn0.2Cu0.2Ag0.2 | 0.012782 |
| 48-148 | Fe0.2In0.2Mn0.2La0.2Cu0.2 | 0.011069 |
| 48-149 | Fe0.2In0.2Mn0.2Sr0.2Ru0.2 | 0.003485 |
| 48-150 | Fe0.2In0.2Mn0.2Sr0.2Ag0.2 | 0.004829 |
| 48-151 | Fe0.2In0.2Mn0.2Sr0.2Cu0.2 | 0.009081 |
| 48-152 | Fe0.2In0.2Mn0.2Pb0.2Ru0.2 | 0.005568 |
| 48-153 | Fe0.2In0.2Mn0.2Pb0.2Ag0.2 | 0.016989 |
| 48-154 | Fe0.2In0.2Mn0.2Pb0.2Cu0.2 | 0.017339 |
| 48-155 | Cr0.2Mn0.2Cu0.2Ag0.2Ru0.2 | 0.002785 |
| 48-156 | Cr0.2Mn0.2Sm0.2Cu0.2Ru0.2 | 0.013213 |
| 48-157 | Cr0.2Mn0.2Sm0.2Cu0.2Ag0.2 | 0.00402 |
| 48-158 | Cr0.2Mn0.2Sr0.2Ag0.2Ru0.2 | 0.003233 |
| 48-159 | Cr0.2Mn0.2Sr0.2Cu0.2Ag0.2 | 0.008184 |
| 48-160 | Cr0.2Mn0.2Sr0.2Sm0.2Cu0.2 | 0.001999 |
| 48-161 | Cr0.2Mn0.2Pb0.2Ag0.2Ru0.2 | 0.002871 |
| 48-162 | Cr0.2Mn0.2Pb0.2Re0.2Ru0.2 | 0.001712 |
| 48-163 | Cr0.2Mn0.2Pb0.2Cu0.2Ru0.2 | 0.004438 |
| 48-164 | Cr0.2Mn0.2Pb0.2Cu0.2Ag0.2 | 0.004148 |
| 48-165 | Cr0.2Mn0.2Pb0.2Cu0.2Re0.2 | 0.002552 |
| 48-166 | Cr0.2Mn0.2Pb0.2Sr0.2Cu0.2 | 0.006212 |
| 48-167 | Cr0.2Mn0.2Pb0.2Sr0.2La0.2 | 0.003787 |
| 48-168 | Cr0.2Mn0.2Mo0.2Ag0.2Ru0.2 | 0.002135 |
| 48-169 | Cr0.2Mn0.2Mo0.2Cu0.2Ag0.2 | 0.004974 |
| 48-170 | Cr0.2Mn0.2Mo0.2Sr0.2Ag0.2 | 0.001893 |
| 48-171 | Cr0.2Mn0.2Mo0.2Sr0.2Cu0.2 | 0.003557 |
| 48-172 | Cr0.2Mn0.2Mo0.2Pb0.2Ag0.2 | 0.002158 |
| 48-173 | Cr0.2Mn0.2Mo0.2Pb0.2Cu0.2 | 0.004687 |
| 48-174 | Cr0.2In0.2Mn0.2Pb0.2Cu0.2 | 0.004038 |
| 48-175 | Cr0.2Fe0.2Pb0.2Sr0.2Ru0.2 | 0.002831 |
| 48-176 | Cr0.2Fe0.2Mn0.2Pb0.2Cu0.2 | 0.002878 |
| 48-177 | Cr0.2Fe0.2Mn0.2Pb0.2Sr0.2 | 100 |
| 48-178 | Co0.2Pb0.2La0.2Ag0.2Ru0.2 | 0.006381 |
| 48-179 | Co0.2Pb0.2Sm0.2Ag0.2Ru0.2 | 0.007932 |
| 48-180 | Co0.2Pb0.2Sm0.2Cu0.2Ru.2 | 0.005721 |
| 48-181 | Co0.2Pb0.2Sm0.2Cu0.2Ag0.2 | 0.002175 |
| 48-182 | Co0.2Pb0.2Sm0.2La0.2Ru0.2 | 0.008775 |
| 48-183 | Co0.2Pb0.2Sr0.2Sm0.2Ru0.2 | 0.008258 |
| 48-184 | Co0.2Mn0.2Re0.2Ag0.2Ru0.2 | 0.002012 |
| 48-185 | Co0.2Mn0.2Cu0.2Ag0.2Ru0.2 | 0.010072 |
| 48-186 | Co0.2Mn0.2Cu0.2Re0.2Ru0.2 | 0.002315 |
| 48-187 | Co0.2Mn0.2Cu0.2Re0.2Ag0.2 | 0.003014 |
| 48-188 | Co0.2Mn0.2La0.2Ag0.2Ru0.2 | 0.008327 |
| 48-189 | Co0.2Mn0.2La0.2Cu0.2Ru0.2 | 0.009596 |
| 48-190 | Co0.2Mn0.2La0.2Cu0.2Ag0.2 | 0.011044 |
| 48-191 | Co0.2Mn0.2Sm0.2Cu0.2Ru0.2 | 0.006625 |
| 48-192 | Co0.2Mn0.2Sm0.2La0.2Ag0.2 | 0.004675 |
| 48-193 | Co0.2Mn0.2Sm0.2La0.2Cu0.2 | 0.005601 |
| 48-194 | Co0.2Mn0.2Sr0.2Ag0.2Ru0.2 | 0.006746 |
| 48-195 | Co0.2Mn0.2Sr0.2Re0.2Ru0.2 | 0.00432 |
| 48-196 | Co0.2Mn0.2Sr0.2Re0.2Ag0.2 | 0.00432 |
| 48-197 | Co0.2Mn0.2Sr0.2Cu0.2Ru0.2 | 0.006853 |
| 48-198 | Co0.2Mn0.2Sr0.2Cu0.2Ag0.2 | 0.007283 |
| 48-199 | Co0.2Mn0.2Sr0.2Cu0.2Re0.2 | 0.00371 |
| 48-200 | Co0.2Mn0.2Sr0.2La0.2Ru0.2 | 0.271262 |
| 48-201 | Co0.2Mn0.2Sr0.2Sm0.2Ag0.2 | 0.006548 |
| 48-202 | Co0.2Mn0.2Pb0.2Ag0.2Ru0.2 | 0.010398 |
| 48-203 | Co0.2Mn0.2Pb0.2Cu0.2Ru0.2 | 0.00966 |
| 48-204 | Co0.2Mn0.2Pb0.2Cu0.2Ag0.2 | 0.015389 |
| 48-205 | Co0.2Mn0.2Pb0.2Cu0.2Re0.2 | 0.002399 |
| 48-206 | Co0.2Mn0.2Pb0.2La0.2Ru0.2 | 0.00918 |
| 48-207 | Co0.2Mn0.2Pb0.2La0.2Ag0.2 | 0.006342 |
| 48-208 | Co0.2Mn0.2Pb0.2La0.2Re0.2 | 0.002061 |
| 48-209 | Co0.2Mn0.2Pb0.2La0.2Cu0.2 | 0.007022 |
| 48-210 | Co0.2Mn0.2Pb0.2Sm0.2Ru0.2 | 0.007602 |
| 48-211 | Co0.2Mn0.2Pb0.2Sm0.2Ag0.2 | 0.00652 |
| 48-212 | Co0.2Mn0.2Pb0.2Sr0.2Ag0.2 | 0.009239 |
| 48-213 | Co0.2Mn0.2Pb0.2Sr0.2Cu0.2 | 0.008812 |
| 48-214 | Co0.2Mn0.2Mo0.2Cu0.2Ru0.2 | 0.003072 |
| 48-215 | Co0.2Mn0.2Mo0.2Cu0.2Ag0.2 | 0.015548 |
| 48-216 | Co0.2Mn0.2Mo0.2La0.2Ag0.2 | 0.002154 |
| 48-217 | Co0.2Mn0.2Mo0.2Pb0.2Ag0.2 | 0.002521 |
| 48-218 | Co0.2Mn0.2Mo0.2Pb0.2Cu0.2 | 0.001672 |
| 48-219 | Co0.2In0.2Pb0.2Ag0.2Ru0.2 | 0.006195 |
| 48-220 | Co0.2In0.2Pb0.2Cu0.2Ag0.2 | 0.003634 |
| 48-221 | Co0.2In0.2Pb0.2La0.2Ru0.2 | 0.006843 |
| 48-222 | Co0.2In0.2Mn0.2Ag0.2Ru0.2 | 0.015644 |
| 48-223 | Co0.2In0.2Mn0.2Cu0.2Ru0.2 | 0.011577 |
| 48-224 | Co0.2In0.2Mn0.2Cu0.2Ag0.2 | 0.011251 |
| 48-225 | Co0.2In0.2Mn0.2La0.2Ag0.2 | 0.014429 |
| 48-226 | Co0.2In0.2Mn0.2La0.2Cu0.2 | 0.00907 |
| 48-227 | Co0.2In0.2Mn0.2Sm0.2Ru0.2 | 0.003887 |
| 48-228 | Co0.2In0.2Mn0.2Sm0.2Ag0.2 | 0.007555 |
| 48-229 | Co0.2In0.2Mn0.2Sm0.2Cu0.2 | 0.005645 |
| 48-230 | Co0.2In0.2Mn0.2Sr0.2Ru0.2 | 0.005456 |
| 48-231 | Co0.2In0.2Mn0.2Sr0.2Ag0.2 | 0.012151 |
| 48-232 | Co0.2In0.2Mn0.2Pb0.2Ru0.2 | 0.008888 |
| 48-233 | Co0.2In0.2Mn0.2Pb0.2Ag0.2 | 0.016836 |
| 48-234 | Co0.2In0.2Mn0.2Pb0.2Cu0.2 | 0.011771 |
| 48-235 | Co0.2In0.2Mn0.2Mo0.2Ag0.2 | 0.003767 |
| 48-236 | Co0.2In0.2Mn0.2Mo0.2Cu0.2 | 0.00326 |
| 48-237 | Co0.2In0.2Mn0.2Mo0.2Sm0.2 | 0.002524 |
| 48-238 | Co0.2In0.2Mn0.2Mo0.2Pb0.2 | 0.003762 |
| 48-239 | Co0.2Fe0.2Pb0.2Ag0.2Ru0.2 | 0.006487 |
| 48-240 | Co0.2Fe0.2Pb0.2Cu0.2Ru0.2 | 0.005269 |
| 48-241 | Co0.2Fe0.2Pb0.2Cu0.2Ag0.2 | 0.003484 |
| 48-242 | Co0.2Fe0.2Pb0.2La0.2Ru0.2 | 0.007154 |
| 48-243 | Co0.2Fe0.2Mn0.2Ag0.2Ru0.2 | 0.016363 |
| 48-244 | Co0.2Fe0.2Mn0.2Cu0.2Ru0.2 | 0.009176 |
| 48-245 | Co0.2Fe0.2Mn0.2Cu0.2Ag0.2 | 0.010893 |
| 48-246 | Co0.2Fe0.2Mn0.2La0.2Ag0.2 | 0.017633 |
| 48-247 | Co0.2Fe0.2Mn0.2La0.2Cu0.2 | 0.008086 |
| 48-248 | Co0.2Fe0.2Mn0.2Sm0.2Ru0.2 | 0.003301 |
| 48-249 | Co0.2Fe0.2Mn0.2Sm0.2Ag0.2 | 0.016937 |
| 48-250 | Co0.2Fe0.2Mn0.2Sm0.2Cu0.2 | 0.006275 |
| 48-251 | Co0.2Fe0.2Mn0.2Sr0.2Ru0.2 | 0.004869 |
| 48-252 | Co0.2Fe0.2Mn0.2Sr0.2Ag0.2 | 0.020005 |
| 48-253 | Co0.2Fe0.2Mn0.2Sr0.2Re0.2 | 0.014097 |
| 48-254 | Co0.2Fe0.2Mn0.2Sr0.2Cu0.2 | 0.00553 |
| 48-255 | Co0.2Fe0.2Mn0.2Pb0.2Ru0.2 | 0.008605 |
| 48-256 | Co0.2Fe0.2Mn0.2Pb0.2Ag0.2 | 0.042132 |
| 48-257 | Co0.2Fe0.2Mn0.2Pb0.2Cu0.2 | 0.011718 |
| 48-258 | Co0.2Fe0.2Mn0.2Mo0.2Cu0.2 | 0.006828 |
| 48-259 | Co0.2Fe0.2Mn0.2Mo0.2Pb0.2 | 0.003513 |
| 48-260 | Co0.2Fe0.2In0.2Ag0.2Ru0.2 | 0.006548 |
| 48-261 | Co0.2Fe0.2In0.2Re0.2Ru0.2 | 0.002563 |
| 48-262 | Co0.2Fe0.2In0.2Sr0.2Ru0.2 | 0.00481 |
| 48-263 | Co0.2Fe0.2In0.2Pb0.2Ru0.2 | 0.006264 |
| 48-264 | Co0.2Fe0.2In0.2Mn0.2Ru0.2 | 0.005415 |
| 48-265 | Co0.2Fe0.2In0.2Mn0.2Ag0.2 | 0.017536 |
| 48-266 | Co0.2Fe0.2In0.2Mn0.2Cu0.2 | 0.010561 |
| 48-267 | Co0.2Cr0.2Sm0.2Ag0.2Ru0.2 | 0.003268 |
| 48-268 | Co0.2Cr0.2Sr0.2Sm0.2Ru0.2 | 0.003364 |
| 48-269 | Co0.2Cr0.2Pb0.2Ag0.2Ru0.2 | 0.003087 |
| 48-270 | Co0.2Cr0.2Pb0.2Sr0.2Ru0.2 | 0.002742 |
| 48-271 | Co0.2Cr0.2Mn0.2Cu0.2Ag0.2 | 0.003568 |
| 48-272 | Co0.2Cr0.2In0.2Sm0.2Ru0.2 | 0.001641 |
| 48-273 | Co0.2Cr0.2In0.2Sr0.2Ru0.2 | 0.002092 |
| 48-274 | Co0.2Cr0.2Fe0.2In0.2Ru0.2 | 0.001801 |
| 48-275 | Ce0.2Pb0.2Cu0.2Ag0.2Ru0.2 | 0.00595 |
| 48-276 | Ce0.2Pb0.2Sm0.2Cu0.2Ru0.2 | 0.002638 |
| 48-277 | Ce0.2Pb0.2Sm0.2La0.2Ru0.2 | 0.003165 |
| 48-278 | Ce0.2Pb0.2Sr0.2Ag0.2Ru0.2 | 0.003156 |
| 48-279 | Ce0.2Pb0.2Sr0.2Re0.2Ru0.2 | 0.002784 |
| 48-280 | Ce0.2Mo0.2Pb0.2Cu0.2Ru0.2 | 0.002798 |
| 48-281 | Ce0.2Mn0.2Pb0.2Ag0.2Ru0.2 | 0.013473 |
| 48-282 | Ce0.2Mn0.2Pb0.2Re0.2Ru0.2 | 0.007226 |
| 48-283 | Ce0.2Mn0.2Pb0.2Cu0.2Ru0.2 | 0.01225 |
| 48-284 | Ce0.2Mn0.2Pb0.2Cu0.2Ag0.2 | 0.025262 |
| 48-285 | Ce0.2Mn0.2Pb0.2Cu0.2Re0.2 | 0.008015 |
| 48-286 | Ce0.2Mn0.2Pb0.2La0.2Ru0.2 | 0.009298 |
| 48-287 | Ce0.2Mn0.2Pb0.2La0.2Ag0.2 | 0.017986 |
| 48-288 | Ce0.2Mn0.2Pb0.2La0.2Cu0.2 | 0.019189 |
| 48-289 | Ce0.2Mn0.2Pb0.2Sm0.2Ru0.2 | 0.007745 |
| 48-290 | Ce0.2Mn0.2Pb0.2Sm0.2Ag0.2 | 0.009861 |
| 48-291 | Ce0.2Mn0.2Pb0.2Sm0.2Cu0.2 | 0.007944 |
| 48-292 | Ce0.2Mn0.2Pb0.2Sm0.2La0.2 | 0.002889 |
| 48-293 | Ce0.2Mn0.2Pb0.2Sr0.2Ru0.2 | 0.006489 |
| 48-294 | Ce0.2Mn0.2Pb0.2Sr0.2Ag0.2 | 0.013705 |
| 48-295 | Ce0.2Mn0.2Pb0.2Sr0.2Cu0.2 | 0.014288 |
| 48-296 | Ce0.2Mn0.2Mo0.2Pb0.2Ag0.2 | 0.014086 |
| 48-297 | Ce0.2Mn0.2Mo0.2Pb0.2Cu0.2 | 0.007669 |
| 48-298 | Ce0.2In0.2Pb0.2Cu0.2Ru0.2 | 0.004894 |
| 48-299 | Ce0.2In0.2Pb0.2La0.2Ru0.2 | 0.003342 |
| 48-300 | Ce0.2In0.2Pb0.2Sm0.2Ru0.2 | 0.003665 |
| 48-301 | Ce0.2In0.2Mn0.2Pb0.2Ru0.2 | 0.007646 |
| 48-302 | Ce0.2In0.2Mn0.2Pb0.2Ag0.2 | 0.014165 |
| 48-303 | Ce0.2In0.2Mn0.2Pb0.2Cu0.2 | 0.020174 |
| 48-304 | Ce0.2Fe0.2Pb0.2Cu0.2Ru0.2 | 0.006186 |
| 48-305 | Ce0.2Fe0.2Pb0.2La0.2Ru0.2 | 0.003994 |
| 48-306 | Ce0.2Fe0.2Pb0.2Sm0.2Ru0.2 | 0.004285 |
| 48-307 | Ce0.2Fe0.2Mn0.2Pb0.2Ru0.2 | 0.004223 |
| 48-308 | Ce0.2Fe0.2Mn0.2Pb0.2Ag0.2 | 0.007856 |
| 48-309 | Ce0.2Fe0.2Mn0.2Pb0.2Cu0.2 | 0.018636 |
| 48-310 | Ce0.2Fe0.2In0.2Pb0.2Ru0.2 | 0.002767 |
| 48-311 | Ce0.2Co0.2Cu0.2Ag0.2Ru0.2 | 0.004839 |
| 48-312 | Ce0.2Co0.2Sm0.2Cu0.2Ru0.2 | 0.004853 |
| 48-313 | Ce0.2Co0.2Sm0.2La0.2Ru0.2 | 0.005343 |
| 48-314 | Ce0.2Co0.2Sr0.2Re0.2Ru0.2 | 0.00375 |
| 48-315 | Ce0.2Co0.2Mo0.2Re0.2Ru0.2 | 0.002159 |
| 48-316 | Ce0.2Co0.2Mo0.2Cu0.2Ru0.2 | 0.00346 |
| 48-317 | Ce0.2Co0.2Mn0.2Ag0.2Ru0.2 | 0.011538 |
| 48-318 | Ce0.2Co0.2Mn0.2Cu0.2Ru0.2 | 0.011344 |
| 48-319 | Ce0.2Co0.2Mn0.2La0.2Ag0.2 | 0.011437 |
| 48-320 | Ce0.2Co0.2Mn0.2La0.2Cu0.2 | 0.006375 |
| 48-321 | Ce0.2Co0.2Mn0.2Sm0.2Ru0.2 | 0.005496 |
| 48-322 | Ce0.2Co0.2Mn0.2Sm0.2Ag0.2 | 0.00495 |
| 48-323 | Ce0.2Co0.2Mn0.2Sm0.2Cu0.2 | 0.005712 |
| 48-324 | Ce0.2Co0.2Mn0.2Sr0.2Ru0.2 | 0.007498 |
| 48-325 | Ce0.2Co0.2Mn0.2Sr0.2Ag0.2 | 0.014609 |
| 48-326 | Ce0.2Co0.2Mn0.2Sr0.2Re0.2 | 0.001438 |
| 48-327 | Ce0.2Co0.2Mn0.2Pb0.2Ru0.2 | 0.011824 |
| 48-328 | Ce0.2Co0.2Mn0.2Pb0.2Ag0.2 | 0.021076 |
| 48-329 | Ce0.2Co0.2Mn0.2Pb0.2Cu0.2 | 0.011215 |
| 48-330 | Ce0.2Co0.2Mn0.2Mo0.2Ag0.2 | 0.003479 |
| 48-331 | Ce0.2Co0.2Mn0.2Mo0.2Cu0.2 | 0.003022 |
| 48-332 | Ce0.2Co0.2Mn0.2Mo0.2Sm0.2 | 0.003106 |
| 48-333 | Ce0.2Co0.2In0.2Ag0.2Ru0.2 | 0.005835 |
| 48-334 | Ce0.2Co0.2In0.2Sr0.2Ru0.2 | 0.004641 |
| 48-335 | Pb0.5Pd0.5 | 0.008444 |
| 48-336 | Fe0.3333Pb0.3333Pd0.3333 | 0.010177 |
| 48-337 | Co0.3333Pd0.3333Ru0.3333 | 0.013571 |
| 48-338 | Co0.3333Rh0.3333Ru0.3333 | 0.004597 |
| 48-339 | Co0.3333Rh0.3333Ag0.3333 | 0.002091 |
| 48-340 | Co0.3333Cs0.3333Pd0.3333 | 0.007353 |
| 48-341 | Ce0.3333Co0.3333Pd0.3333 | 0.007465 |
| 48-342 | Bi0.25Pd0.25Ag0.25Ru0.25 | 0.003244 |
| 48-343 | Bi0.25Pd0.25Cu0.25Ru0.25 | 0.003825 |
| 48-344 | Bi0.25Rh0.25Cu0.25Ru0.25 | 0.010849 |
| 48-345 | Pb0.25Pd0.25Ag0.25Ru0.25 | 0.005382 |
| 48-346 | Pb0.25Pd0.25Cu0.25Ru0.25 | 0.00449 |
| 48-347 | K0.25Bi0.25Rh0.25Ru0.25 | 0.003768 |
| 48-348 | K0.25Bi0.25Rh0.25Cu0.25 | 0.003595 |
| 48-349 | K0.25Pb0.25Rh0.25Ru0.25 | 0.003963 |
| 48-350 | K0.25Pb0.25Rh0.25Ag0.25 | 0.003113 |
| 48-351 | Mn0.25Bi0.25Pd0.25Ag0.25 | 0.006105 |
| 48-352 | Mn0.25Bi0.25Pd0.25Cu0.25 | 0.009397 |
| 48-353 | Mn0.25Bi0.25Rh0.25Ru0.25 | 0.004306 |
| 48-354 | Mn0.25Bi0.25Rh0.25Ag0.25 | 0.003965 |
| 48-355 | Mn0.25Bi0.25Rh0.25Cu0.25 | 0.01056 |
| 48-356 | Mn0.25Pb0.25Pd0.25Ru0.25 | 0.008161 |
| 48-357 | Mn0.25Pb0.25Pd0.25Ag0.25 | 0.014522 |
| 48-358 | Mn0.25Pb0.25Pd0.25Cu0.25 | 0.016751 |
| 48-359 | Mn0.25Pb0.25Rh0.25Ru0.25 | 0.006114 |
| 48-360 | Mn0.25Pb0.25Rh0.25Cu0.25 | 0.014948 |
| 48-361 | Nd0.25Bi0.25Rh0.25Ru0.25 | 0.002901 |
| 48-362 | Nd0.25Bi0.25Ru0.25Cu0.25 | 0.002956 |
| 48-363 | Nd0.25Pb0.25Pd0.25Ru0.25 | 0.010621 |
| 48-364 | Nd0.25Pb0.25Rh0.25Ru0.25 | 0.00452 |
| 48-365 | Nd0.25Mn0.25Pb0.25Pd0.25 | 0.010304 |
| 48-366 | Nd0.25Mn0.25Pb0.25Rh0.25 | 0.00301 |
| 48-367 | Fe0.25Bi0.25Rh0.25Ru0.25 | 0.002977 |
| 48-368 | Fe0.25Bi0.25Rh0.25Cu0.25 | 0.003752 |
| 48-369 | Fe0.25Pb0.25Rh0.25Ru0.25 | 0.002039 |
| 48-370 | Fe0.25Pb0.25Rh0.25Ag0.25 | 0.004077 |
| 48-371 | Fe0.25Mn0.25Pb0.25Pd0.25 | 0.004793 |
| 48-372 | Fe0.25Nd0.25Pb0.25Pd0.25 | 0.012943 |
| 48-373 | Cs0.25Pb0.25Pd0.25Ag0.25 | 0.00941 |
| 48-374 | Cs0.25Pb0.25Rh0.25Ru0.25 | 0.003881 |
| 48-375 | Cs0.25Pb0.25Rh0.25Ag0.25 | 0.002395 |
| 48-376 | Cs0.25Pb0.25Rh0.25Cu0.25 | 0.002333 |
| 48-377 | Cs0.25Mn0.25Bi0.25Rh0.25 | 0.002735 |
| 48-378 | Ni0.5Ag0.5 | 0.020998 |
| 48-379 | Mn0.3333Pb0.3333W0.3333 | 0.00382 |
| 48-380 | Ni0.3333Ag0.3333Ru0.3333 | 0.002507 |
| 48-381 | Ni0.3333Tl0.3333Ag0.3333 | 0.010436 |
| 48-382 | Ni0.3333Y0.3333Ag0.3333 | 0.002472 |
| 48-383 | Ni0.3333Mn0.3333Ru0.3333 | 0.007979 |
| 48-384 | Ni0.3333Mn0.3333Ag0.3333 | 0.002857 |
| 48-385 | Ni0.3333Mn0.3333Cu0.3333 | 0.002609 |
| 48-386 | Ni0.3333Mn0.3333Tl0.3333 | 0.002267 |
| 48-387 | Ni0.3333Mn0.3333Pb0.3333 | 0.003725 |
| 48-388 | Ni0.3333Er0.3333Ag0.3333 | 0.008514 |
| 48-389 | Ni0.3333Eu0.3333Ag0.3333 | 0.008206 |
| 48-390 | Ni0.3333Ba0.3333Ag0.3333 | 0.003011 |
| 48-391 | Ni0.3333Co0.3333Ag0.3333 | 0.009938 |
| 48-392 | Mn0.25W0.25Cu0.25Nb0.25 | 0.003392 |
| 48-393 | Mn0.3333Pb0.1111Tl0.5556 | 0.055669 |
| 48-394 | Mn0.4544Pb0.0909Tl0.4544 | 0.040482 |
| 48-395 | Mn0.4167Pb0.0833Tl0.4167Ag0.0833 | 0.046529 |
| 48-396 | Mn0.3751Pb0.1251Tl0.3751Cu0.1251 | 0.045131 |
| 48-397 | Mn0.3Pb0.1Tl0.3Cu0.3 | 0.046667 |
| 48-398 | Mn0.25Pb0.0833Tl0.25Cu0.4167 | 0.04952 |
| 48-399 | Mn0.5Pb0.1Tl0.3Cu0.1 | 0.041957 |
| 48-400 | Mn0.4167Pb0.0833Tl0.25Cu0.25 | 0.041416 |
| 48-401 | Mn0.2142Pb0.2142Tl0.2142Cu0.3571 | 0.042629 |
| 48-402 | Mn0.4167Pb0.25Tl0.25Cu0.0833 | 0.043314 |
| 48-403 | Mn0.3Pb0.1Tl0.5Cu0.1 | 0.042344 |
| 48-404 | Mn0.4167Pb0.0833Tl0.4167Cu0.0833 | 0.047881 |
| 48-405 | Mn0.3571Pb0.0713Tl0.3571Cu0.2142 | 0.049461 |
| 48-406 | Mn0.3124Pb0.0624Tl0.3124Cu0.3124 | 0.049975 |
| 48-407 | Mn0.2778Pb0.1667Tl0.2778Cu0.2778 | 0.040557 |
| 48-408 | Cs0.1251Mn0.3751Pb0.1251Tl0.3751 | 0.06866 |
| 48-409 | Cs0.1Mn0.3Pb0.1Tl0.5 | 0.045689 |
| 48-410 | Cs0.1Mn0.5Pb0.1Tl0.3 | 0.046045 |
| 48-411 | Cs0.0833Mn0.4167Pb0.0833Tl0.4167 | 0.069696 |
| 48-412 | Cs0.3Mn0.5Pb0.1Tl0.1 | 0.044708 |
| 48-413 | Cs0.25Mn0.4167Pb0.0833Tl0.25 | 0.040287 |
| 48-414 | Cs0.3571Mn0.3571Pb0.2142Tl0.0713 | 0.07157 |
| 48-415 | Cs0.3124Mn0.3124Pb0.1876Tl0.1876 | 0.054371 |
| 48-416 | Mn0.3333Pb0.1111Tl0.1111Cu0.1111Ag0.3333 | 0.043155 |
| 48-417 | Mn0.2727Pb0.0909Tl0.0909Cu0.0909Ag0.4544 | 0.051458 |
| 48-418 | Mn0.3847Pb0.0769Tl0.0769Cu0.0769Ag0.3847 | 0.055245 |
| 48-419 | Mn0.3333Pb0.2Tl0.0667Cu0.0667Ag0.3333 | 0.040265 |
| 48-420 | Mn0.4544Pb0.0909Tl0.2727Cu0.0909Ag0.0909 | 0.044675 |
| 48-421 | Mn0.3333Pb0.0667Tl0.2Cu0.0667Ag0.3333 | 0.05226 |
| 48-422 | Mn0.3847Pb0.0769Tl0.3847Cu0.0769Ag0.0769 | 0.041187 |
| 48-423 | Mn0.2727Pb0.0909Tl0.2727Cu0.2727Ag0.0909 | 0.042675 |
| 48-424 | Mn0.2942Pb0.0589Tl0.1764Cu0.1764Ag0.2942 | 0.040998 |
| 48-425 | Mn0.2307Pb0.0769Tl0.2307Cu0.3847Ag0.0769 | 0.041711 |
| 48-426 | Mn0.238Pb0.0476Tl0.238Cu0.238Ag0.238 | 0.042432 |
| 48-427 | Cs0.0589Mn0.2942Pb0.1764Tl0.2942Ag0.1764 | 0.0403 |

TABLE VII
Examples of ZrO₂-supported catalysts

| | | Propene to |
|---|---|---|
| | | PO |
| | | conversion |
| Example | Composition | [%] |
| 48-428 | Sr0.5Ru0.5 | 0.001858 |
| 48-429 | Mn0.5Ru0.5 | 0.004011 |
| 48-430 | Mo0.3333Pb0.3333Ag0.3333 | 0.016683 |
| 48-431 | Ce0.3333Sr0.3333Ru0.3333 | 0.002675 |

TABLE VIII
Examples of CaCO₃-supported catalysts

| | | Propene to |
|---|---|---|
| | | PO |
| | | conversion |
| Example | Composition | [%] |
| 48-432 | Mn0.5Ag0.5 | 0.00292 |
| 48-433 | Sr0.3333Sm0.3333Cu0.3333 | 0.003056 |
| 48-434 | Mn0.3333La0.3333Ag0.3333 | 0.002239 |
| 48-435 | Mn0.3333Pb0.3333Sm0.3333 | 0.003735 |
| 48-436 | Co0.3333Sm0.3333Ag0.3333 | 0.002252 |

TABLE IX
Examples of SiC-supported catalysts

| | | Propene to |
|---|---|---|
| | | PO |
| | | conversion |
| Example | Composition | [%] |
| 48-437 | Mn0.5Pb0.5 | 0.004456 |
| 48-438 | Mn0.3333Pb0.3333Cu0.3333 | 0.009548 |
| 48-439 | Co0.3333Mn0.3333Ru0.3333 | 0.055461 |
| 48-440 | Mn0.3333Sm0.3333Ru0.3333 | 0.004861 |
| 48-441 | Mn0.3333Sm0.3333Ag0.3333 | 0.002729 |
| 48-442 | Mn0.3333Sm0.3333Cu0.3333 | 0.004519 |

TABLE X
Examples of SiO₂-supported catalysts

| | | Propene to |
|---|---|---|
| | | PO |
| | | conversion |
| Example | Composition | [%] |
| 48-443 | Ag0.5Ru0.5 | 0.002933 |
| 48-444 | Cu0.5Ru0.5 | 0.004538 |
| 48-445 | Pb0.5Ag0.5 | 0.002716 |
| 48-446 | Mn0.5Ru0.5 | 0.004523 |
| 48-447 | Mn0.5Ag0.5 | 0.005384 |
| 48-448 | Mn0.5Cu0.5 | 0.001522 |
| 48-449 | Ce0.5Ru0.5 | 0.010574 |
| 48-450 | Pb0.3333Cu0.3333Ru0.3333 | 0.002703 |
| 48-451 | Mn0.3333La0.3333Ru0.3333 | 0.002698 |
| 48-452 | Mn0.3333Sm0.3333Ru0.3333 | 0.002833 |
| 48-453 | In0.3333Mn0.3333Ru0.3333 | 0.005021 |
| 48-454 | In0.3333Mn0.3333Ag0.3333 | 0.002989 |
| 48-455 | In0.3333Mn0.3333Cu0.3333 | 0.002471 |
| 48-456 | Cr0.3333In0.3333Cu0.3333 | 0.028075 |
| 48-457 | Co0.3333Pb0.3333Ag0.3333 | 0.074228 |
| 48-458 | Co0.3333Fe0.3333Pb0.3333 | 0.125742 |
| 48-459 | Ce0.3333Cu0.3333Ag0.3333 | 0.125893 |
| 48-460 | Ce0.3333La0.3333Ru0.3333 | 0.005351 |
| 48-461 | Ce0.3333Sm0.3333Ru0.3333 | 0.0018 |
| 48-462 | Ce0.3333Sr0.3333Cu0.3333 | 0.026777 |
| 48-463 | Ce0.3333Pb0.3333Ag0.3333 | 0.054395 |
| 48-464 | Ce0.3333In0.3333Ru0.3333 | 0.021632 |
| 48-465 | Ce0.3333Fe0.3333In0.3333 | 0.019295 |
| 48-466 | Ce0.3333Co0.3333Ru0.3333 | 0.219737 |
| 48-467 | Ce0.3333Co0.3333La0.3333 | 0.043075 |

TABLE XI
Example of a TiO₂-supported catalyst

| | | Propene to PO |
|---|---|---|
| Example | Composition | conversion [%] |
| 48-468 | Fe0.3333Re0.3333Ag0.3333 | 0.02037 |

TABLE XII
Examples of SiO₂—TiO₂-supported catalysts

| | | Propene to PO |
|---|---|---|
| Example | Composition | conversion [%] |
| 48-469 | Sr0.5Ru0.5 | 0.003813 |
| 48-470 | Co05Cu0.5 | 0.001745 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A catalyst containing a mixture of at least one element selected from the group consisting of Sc, Y, Ti, Zr, Hf, V, Nb, Ta, Cr, Mo, W, Re, Fe, Co, Ni, Sn, Pb, Sb, Bi, Se and Zn and at least one element selected from the group consisting of Cu, Ru, Rh, Pd, Os, Ir, Pt, Au, In, Tl, Mn and Ce, the mixture being on a porous support.

2. The catalyst according to claim 1, wherein the support has a BET surface area of less than 200 m²/g.

3. The catalyst according to claim 1, wherein the support contains at least one member selected from the group consisting of Al₂O₃, CaCO₃, ZrO₂, SiO₂, SiC, TiO₂ and SiO₂—TiO₂.

4. The catalyst according to claim 3, wherein the support consists of at least one of Al₂O₃, CaCO₃, ZrO₂, SiO₂, SiC, TiO₂ and SiO₂—TiO₂ mixed oxide.

5. The catalyst according to claim 1, wherein the choice of elements from the two groups is made such that the mixture is selected from the group consisting of Bi—Rh, Bi—Ru, Cr—Cu, Cr—Ru, Fe—Ru, Fe—Tl, Fe—Cu, Sb—Ru, Sb—Cu, Ni—Ru, Mo—Cu, Ni—Rh, Ru—Re, Co—Ru, Co—Tl, Mn—Pb, Mn—Cu—Ag—Pb—In, Mn—Cu—Ag—Pb—Sr, Mn—Cu—Ag—Pb, Mn—Pb—Cu—Ru, Mn—Ru—Co—Ba, Eu—Ag—Ni—Tl, Mn—Cu—Ag—Zn, Mn—Ni—Ag—Pb, Mn—Pb—La—Cu, In—Mn—Pb—Ag, Mn—Co—Ag—Pb, Cs—Mn—Pb—Tl, Mn—Pb—Tl—Cu—Ag, Mn—Pb—Tl—Cu, Cs—Mn—Pb—Tl—Ag, Mn—Cu—Pb, Mn—Pb—Ag—Ru, Co—Mn—Pb—Cu—Ag, Co—Fe—Mn—Pb—Ag, Ce—Co—Mn—Pb—Ag, Co—In—Mn—Pb—Ag, Ce—In—Mn—Pb—Cu, and any combination thereof.

6. A process for preparing the catalyst according to claim 1, comprising
preparing the support,
combining the support with a solution containing at least one element selected from the group consisting of Sc, Y, Ti, Zr, Hf, V, Nb, Ta, Cr, Mo, W, Re, Fe, Co, Ni, Sn, Pb, Sb, Bi, Se and Zn and at least one element selected from the group consisting of Cu, Ru, Rh, Pd, Os, Ir, Pt, Au, In, Tl, Mn and Ce, whereby a support loaded with the elements is obtained, and
calcining the support loaded with the elements at a temperature of from 200 to 1000° C.

7. The process according to claim 6, wherein the support is combined with the solution such that the volume of the solution is less than or at most equal to the pore volume of the support.

8. The process according to claim 6, wherein drying is carried out before the calcination.

9. The process according to claim 6, wherein reduction is carried out after the calcination.

10. The catalyst obtained by the process according to claim 6.

11. In a method for the epoxidation of hydrocarbons, the improvement comprising including the catalyst of claim 1.

12. A process for the epoxidation of hydrocarbons with oxygen in the presence of the catalyst according to one of claims 1 and 10.

13. The process according to claim 12, wherein the hydrocarbon is selected from the group consisting of propene and butene.
